(19) United States
(12) Patent Application Publication
(10) Pub. No.: US 2011/0319370 A1
(43) Pub. Date: Dec. 29, 2011

(54) Combination Hormone Therapy for the Eye

(75) Inventors: Moji Christianah Adeyeye, Schaumburg, IL (US); Paula A Witt-Enderby, Bethel Park, PA (US); Vicki L. Davis, Seven Fields, PA (US); Udaya K. Kotreka, Cockeysville, MD (US)

(73) Assignee: DUQUESNE UNIVERSITY OF THE, Pittsburgh, PA (US)

(21) Appl. No.: 13/169,658

(22) Filed: Jun. 27, 2011

Related U.S. Application Data
(60) Provisional application No. 61/398,594, filed on Jun. 28, 2010.

(51) Int. Cl. A61K 31/4045; A61K 31/565; A61P 27/02; A61K 31/568

(52) U.S. Cl. 514/170; 514/415; 514/171

(57) ABSTRACT

An ophthalmic instillation composition which contains melatonin and at least one other hormone, typically an estrogen but also possibly testosterone or another aromatasable androgen. The instillation is designed to reduce or eliminate cataracts in men or women to which such an ophthalmic instillation composition is administered.

### CROSS-REFERENCE TO RELATED APPLICATION

This patent application claims priority to U.S. Provisional Patent Application No. 61/398,594 filed 28 Jun. 2010, which is incorporated herein by reference in its entirety.

### BACKGROUND OF THE INVENTION

1. Field of the Invention

The invention relates to an ophthalmic instillation composition containing at least two hormones which together reduce oxidative stress and even free radical damage in the eye.

2. Description of Related Art

The idea of using estrogen to protect lenses of the eye from cataract formation was published at least as early as 1997, in Hales, A., et al., “Estrogen Protects Lenses against Cataract Induced by Transforming Growth Factor-β (TGFβ),” J. Exp. Med., The Rockefeller University Press, Vol. 185, pp. 273-280, January 1997. The application of melatonin to the eye has also been known since at least as early as Ohanness, A. K., et al., “Protective Effects of Melatonin as an Eye Drops [sic] Against Selenite-Induced Cataract in Rat Pups,” Saudi Pharmaceutical Journal, Vol. 17, No. 2, April, 2009, pp. 148-153. However, there are challenges in implementing either hormone administration which include, without limitation, formulation, toxicity, effectiveness, half life and controlled or sustained release issues. Also, men are in need of cataract reduction or prevention but the acceptability of estrogen-containing compositions to men is generally low. Finally, the benefits of co-administering melatonin with another important hormone have never been considered before, to the knowledge of the inventors. A need thus remains for a composition that is effective, safe, and susceptible of high compliance with male and female patients alike as an instillation ophthalmic composition to reduce or eliminate cataracts or other products of oxidative stress in the eye in a meaningful way.

### SUMMARY OF THE INVENTION

In order to meet this need, the present invention is an ophthalmic instillation composition which contains melatonin and at least one other hormone, typically an estrogen but also possibly testosterone or another aromatasable androgen. Testosterone or other aromatasable androgens convert, upon the action of aromatase in the eye, to estrogen in situ. The estrogen may be without limitation 17β-estradiol, ethinyl estradiol, estrone or estriol. When the composition contains melatonin and at least one other hormone selected from the group consisting of an estrogen or an aromatasable androgen, and the composition is instilled regularly into a patient via ophthalmic administration, the composition is surprisingly effective in controlling or even stopping oxidative stress or free radical damage in the eye and thus reducing cataracts or age-related macular degeneration, among other oxidative stress-caused conditions.

### DETAILED DESCRIPTION OF THE INVENTION

Cataracts commonly occur in the elderly. Due to the lack of therapies to prevent their occurrence or delay their progression, the surgical removal of the lens has heretofore been required to prevent blindness. The rise in the incidence of age-related cataracts coincides with age when the levels of oxidative stress increase, nocturnal melatonin levels decrease, and, in women, estrogen levels decline. Melatonin is a hormone produced in the absence of light (darkness) that has potent antioxidant properties and inhibits cataracts in rat models (Ohanness, A. K., et al., supra). Estrogen also has strong antioxidant properties and is associated with a lower risk of cataracts in women and cataract prevention in rodent models. Both hormones have receptors as well as enzymes for their synthesis in the eye, all of which suggests that they each have important protective functions when present as synthesized naturally.

Age-related cataract incidence is higher in women than in men. In a “Beaver Dam Eye Study,” the prevalence of cataracts was 3.9% in men and 10% in women for ages 55-64. Ovarian failure that occurs after menopause results in women having low estrogen levels starting on average around age 52. The postmenopausal years correspond to ages when cataract risk increases. Several epidemiological studies suggest that different types of age-related cataracts may be influenced by estrogens at different ages of exposure. The Beaver Dam Eye Study reported a modest protective effect of lifetime estrogen exposure on cataract risk. That is, a later onset of menopause and a younger age at menarche (onset of menstruation) are associated with a decreased risk of cataracts. An increased prevalence of cataracts was reported with later ages at menarche and earlier onset of menopause, which suggest that a shorter lifetime exposure to estrogen may increase the risk of age-related cataracts. Another study reported that women with less exposure to endogenous estrogens had three times the risk of age-related cataracts. Hormone replacement therapy in postmenopausal women has been associated with a lower prevalence of nuclear cataracts. These data strongly suggest that estrogen has a protective role on lens transparency in humans.

With age, the risk of cataracts increases, but nocturnal melatonin levels decrease. The increase in cataract risk with age coincides with timing of reduced nocturnal melatonin levels. The decline in nocturnal melatonin also coincides with the ages at which our defenses against oxidative stress are weakened, and oxidative stress is linked to cataracts. These correlations suggest that melatonin protects lens transparency. Although human studies have not examined the role of melatonin for cataract prevention, several preclinical models have demonstrated its protective influence on the lens. In newborn rats treated with buthionine sulfoximine (BSO), cataracts develop by age 16 days and administration of melatonin reduced cataract incidence from 100% to 6.2%. As BSO severely depletes Glutathione (reduced form) (GSH) levels, oxidative stress plays an important role in cataractogenesis in this model, and thus the prevention of lens opacity suggests that melatonin acts as a free radical scavenger and/or by increasing GSH levels. Also, ophthalmic melatonin is beneficial for preventing age-related macular degeneration and glaucoma based on its antioxidative actions and the role of oxidative stress in these diseases.

Using an in situ ophthalmic delivery system, to replace the declining levels of both hormones, protects the lens to prevent the loss of transparency that occurs in aging women and men, and the combination of both hormones (melatonin plus the additional estrogen or aromatasable androgen) reduces caratacts or other products of oxidative stress in the eye better than either hormone alone. In particular, because melatonin suppresses aromatase activity, the enzyme responsible for in situ estrogen synthesis in the eye, the supplementing of an ophthalmic melatonin composition with additional estrogen makes sense to counteract any action of aromatase on estrogen present in the eye. By supplementing both hormones, the invention achieves high levels of protection against oxidative stress and ensures sufficient levels of estrogen as well as melatonin in the eye, for their mutual transparency protective actions in the lens.

The ophthalmic instillation composition contains melatonin and at least one other hormone, generally an estrogen or an aromatasable androgen such as testosterone. A first embodiment of the invention includes 17β-estradiol and melatonin in a saline or other solution or suspension suitable for administration to the eye. However, testosterone (or any other aromatasable androgen) may be substituted in whole or in part for the estrogen. Upon the action of aromatase, testosterone or any other aromatasable androgen converts to estrogen in situ. The partial presence of testosterone or other aromatasable androgen creates a chemical controlled release of estrogen; the complete substitution of testosterone (or other androgen) for estrogen creates a composition likely to be more acceptable to men who desire cataract reduction but whom are wary about accepting dosage forms containing female hormones even when they are simply for ophthalmic administration.

Diluents, excipients and carriers for the combined hormones may be any known in the art. The use of thickeners such as polymers that increase the residence time of the hormones in the eye are preferred, but the invention embraces any ophthalmic instillation composition containing melatonin and at least one other estrogen or aromatasable androgen, in the presence of any suitable carrier, solubilizing agent or diluent. These diluents, solubilizing agents, excipients and carriers include, without limitation: aqueous sodium chloride; aqueous sodium alginate; isotonic borate buffer; aqueous calcium chloride; gellan gum; carbopol; guar gum; hydroxypropylmethylcellulose (HPMC) and other cellulose derivatives common in the pharmaceutical industry; petrolatum suspension; free fatty acids in aqueous or aqueous saline suspension and aqueous benzalkonium chloride. Gellan-gum-containing polymers are typically used in in situ gels; compositions that do not form in situ gels may be made from HPMCs, for example. Isotonic borate buffer may be prepared according to, for example, “borax buffer according to Palitzsch” in OPHTHALMICA, Volume 1, Pharmazeutische Grundlagen and ihre Zubereitung, Wissenschaftliche Verlagsgesellschaft mbH, Stuttgart, page 95, Table 3.2/7, (1975).

When an in situ gel is desired, the following information is relevant. Dependent upon pH, ionic contents and temperature present in the eye, the gel can instantaneously form upon dropwise topical administration to the eye. Deacetylated Gellan gum (Kelcogel F or CG-LA or CG-HA and different proportions of CG-LA and CG-HA in admixture) is an anionic polysaccharide and offers gelation upon ocular administration in the presence of ionic contents of the tear fluid. It offers both the advantages of forming low viscosity clear preparations in vitro as well as strong in situ gelation upon ocular administration. The chemical structure is a tetrasaccharide repeating unit which has been well characterized to date. Gellan gum in solutions forms a coaxial triangular 3-fold double helix from two left-handed chains coiled around each other with the acetate residues on the periphery and glyceryl groups stabilizing the inter-chain associations to form a gel network in the presence of cations present in the eye. Hydrogen-bonds are formed between the hydroxy methyl of 4-linked glucosyl units of one chain and the carboxylate group of other. Ion-binding sites are provided by both carboxylate oxygen atoms and a hydroxyl group in one chain and two hydroxyl groups in the other plus one strongly-bound water molecule.49-50. Drug delivery to ocular mucosa for local treatment or systemic effect is associated with great possibilities, but often also with many obstacles. The physiological constraints imposed by the protective mechanisms of the eye (such as the corneal barrier comprised of the epithelium, endothelium and the inner stroma), as well as short pre-corneal residence time of typical ophthalmic solutions due to constant lachrymal drainage, lead to low absorption of drugs and short duration of therapeutic effect. Tear fluid also contributes to lower bioavailability by washing away the drug due to the body's reflex defense mechanism (about 5% or less) and thus requires frequent administration. Moreover, systemic absorption of the drug drained through the naso-lachrymal duct may result in some undesirable side effects. Creation of in situ gels can address some of these concerns, as is known in the art.

Solubility and stability of the active agents in the solution or suspension of the present invention may be addressed by means known in the art at this writing. The solubility of the drug and stability in the aqueous medium are important determinants for successful formulation of an ophthalmic delivery system. Solubility, viscosity (in Centipoise), lipophilicity, partition coefficient (K) and molecular size (D) influence the flux of the drug across the corneal epithelium with a fixed thickness (h), as represented by Fick's law or J=DKCs/h. The flux determines the permeation across the membrane and bioavailability. Drugs with low water solubility and high permeability such as 17β-estradiol and melatonin belong to the biopharmaceutical classification (BCS) Class II. Estradiol is practically insoluble in water, with a solubility of 0.03 mg/dl at 25° C. Melatonin is soluble in organic solvents (140.5 mg/ml) and scantily soluble in aqueous media (solubility of 2.4 mg/ml). Because the solubility of the two drugs is low, other excipients must be incorporated into the formulation to enhance solubilization or prevent recrystallization upon storage. Solubilizers such as cyclodextrins, surfactants (hydrophilic) and mixed solvents are often used to achieve this goal. Compatibility of the solubilizers with other excipients and drugs must also be established. It is important that any delivery system be characterized for the stability of the drugs. This is essential for prediction of shelf life, ruling out formation or identification of degradative products and establishing safety of the drug product for human consumption. In addition, according to International Conference on Harmonization (ICH) guidelines, stress testing of the drug substance can help identify the likely degradation products which can in turn help establish the degradation pathways and the intrinsic stability of the molecule and validate the stability indicating power of the analytical procedures used.

When an in situ gel is used as discussed above, the in situ gel formulation should prolong residence or contact time on the eye surface. The drug then partitions through the epithelium and is slowly released into the corneal stroma and further into the anterior chamber (20-30 mins). Melatonin and 17β-estradiol, due to their lipophilicity, permeate relatively faster into the anterior chamber (aqueous humor) from where the drug can access the iris and the ciliary body. Clearance from the aqueous humor occurs due to turnover in the chamber or penetration across the endothelial cells of the uveal wall into the systemic circulation. This process is favored by the lipophilicity of the two drugs, and melatonin and 17β-estradiol permeate quickly into the anterior chamber and thus also quickly reach the inside of the lens after administration.

Dosing of the two combined hormones should take into account the following. 17β-estradiol and melatonin should be formulated at a concentration of about 2 μg-250 μg/ml for the estradiol and about 2-200 mg/ml for the melatonin, dissolving the active agents with the aid of solubilizers. By “about” is meant plus or minus ten percent. Dosing is dropwise as needed, with about a 25-50 μg dose one to three times per day. When testosterone or another aromatasable androgen is substituted in whole or in part for the estradiol—or when another estrogen is used—the total amount of hormone should be about the same as when the estradiol alone is used.

In general, the amount of estrogen present in an ophthalmic composition intended to reduce or eliminate cataracts may be present at the lower end of the range of estrogen—down to about half that amount—when melatonin is present. For example, in a formulation containing sterile deionized water, 0.001-0.01% w/v of an estrogen compound (such as without limitation 17β-estradiol, ethinyl estradiol, estrone or estriol); 0.01-4% w/v of an agent capable of solubilizing and complexing estrogen compounds, preferably either 0.5-4% w/v nonionic surfactant Polysorbate 80 (a well known nonionic surfactant and emulsifier derived from polyethoxylated sorbitan and oleic acid) or 0.01-1% w/v cyclodextrin; optional 2-3% w/v glycerin; 0.1-0.4% w/v sorbic acid or potassium, calcium or sodium sorbate as preservative; optionally about 0.01-0.1% ethylenediamine tetraacetic acid (EDTA) or disodium edetate dehydrate; mannitol about 4-5% w/v if the optional glycerin is not present; and gellan gum 0.1-0.5% w/v as an in-situ gel forming polymer vehicle—the estradiol inclusion could be reduced to about 0.0005-0.001% w/v if melatonin is also present in approximate amounts as disclosed elsewhere herein. Having said that, however, the estrogen amount does not have to be limited to the lower end of the range for some formulations, because some patients may need higher estrogen levels even when they are treated with the estrogen (or other hormone as discussed herein) and melatonin combined. When low estrogen levels are required or desired, such as by men or by women at risk for breast cancer, the addition of melatonin gives strong protection when combined with the doses for estrogen near the lower end or even below (estimated up to half to the lowest dose in the range) to prevent altering the patient's baseline circulating estrogen levels. On the other hand, if estrogen is given alone, it is likely to be protective in many women and men. However, the levels delivered to the eye that avoid systemic absorption and possible adverse effects in turn may not provide as sufficient protection for cataracts associated with higher oxidative stress levels, such as with diabetes, smoking, and radiation. Since melatonin also has strong antioxidant activity and little to no toxicity, systemic absorption does not need to be limited and may even be desired, so higher levels can be delivered to the eye to allow free radical scavenging as well as antioxidant protection. Therefore, the combination of estradiol plus melatonin in an ophthalmic delivery system has more applications and potential benefits against multiple risk factors linked to cataracts than the administration of either hormone alone. Stated a different way, although estrogen may be protective in postmenopausal women, most women have at least one other risk factors that may also contribute to cataract development, such as smoking, UV exposure, or diabetes, so that providing another layer of protection by supplementing both estrogen and melatonin in the eye is a significant health improvement for virtually all women as well as many men.

The following Example is illustrative.

### EXAMPLE 1

Development of fixed dose combination of 17β estradiol and melatonin in an in situ gel eye drop formulation proceeds as follows. The in situ gel solution (100 ml) is prepared using ion-activation in situ gelation in which mono (Na+, K+) and divalent (Ca++) cations present in physiological fluids cause gelation of the solution upon instillation into the eye. Deacetylated gellan gum, an anionic polysaccharide, is dispersed in deionized water containing osmotic agent and preservatives at 90° C. to form a clear solution that is then allowed to cool to room temperature while stirring overnight. The solution is then autoclaved at 121° C. for 20 minutes. A baseline viscosity of the solution is determined using Texas AR 1000 Rheometer viscometer for reference. Sterility of the gum is tested and confirmed according to USP anti-microbial effectiveness specifications. A baseline clarity is also determined for comparison to stored samples by UV spectrometer.

The drugs (estradiol and melatonin or melatonin alone) in concentrations equivalent to 2 m-250 m/ml for estradiol and 2-200 mg/ml for melatonin are dissolved using a solubilizer, sterilized by filtration under aseptic condition using 0.22 μm sterilizing filter, and mixed with other formulation contents for 30 minutes under aseptic conditions. All batches are tested for pH, potency, clarity, osmolarity, drug release, viscoelasticity and acute eye irritation.

Although the technology of the invention has been described with particularity above, the invention itself is only to be considered to be limited insofar as is set forth in the accompanying claims.

1. A composition for ophthalmic instillation in the eye, comprising a quantity of melatonin and a quantity of a second hormone in a pharmaceutically acceptable carrier.

2. The composition of claim 1, wherein said second hormone is selected from the group consisting of an estrogen or an aromatasable androgen.

3. The composition of claim 1, wherein said second hormone is estradiol.

4. The composition of claim 1, wherein melatonin, estradiol and testosterone are present.
The composition of claim 1, wherein said second hormone is testosterone.
